(19) United States
(12) Patent Application Publication
(10) Pub. No.: US 2024/0245603 A1
(43) Pub. Date: Jul. 25, 2024

(54) HIGH LOADING ORAL FILM FORMULATION

(71) Applicant: INTELGENX CORP., St-Laurent (CA)

(72) Inventors: Billal TIR, Montréal (CA); Nadine PAIEMENT, St-Laurent (CA)

(73) Assignee: Intelgenx Corp., St-Laurent, QC (CA)

(21) Appl. No.: 18/275,610

(22) PCT Filed: Feb. 7, 2022
(86) PCT No.: PCT/CA2022/050171

§ 371 Date: Aug. 3, 2023

Related U.S. Application Data
(60) Provisional application No. 63/146,458, filed on Feb. 5, 2021.
(60) Provisional application No. 63/146,706, filed on Feb. 7, 2021.

(51) Int. Cl. A61K 9/00; A61K 31/439

(52) U.S. Cl. CPC A61K 9/006; A61K 31/439

(57) ABSTRACT

An oral film dosage form includes a high loading of API designed to be administered in some form to a human subject and in some other form to an animal subject.

### CROSS-REFERENCE TO RELATED APPLICATIONS

The present application claims priority to U.S. Provisional Patent Application No. 63/146,458, which was filed Feb. 5, 2021 and U.S. Provisional Patent Application No. 63/146,706, which was filed Feb. 7, 2021, the contents of which are incorporated herein by reference in their entirety.

### FIELD OF THE DISCLOSURE

This disclosure relates to oral film dosage formulations and processes for preparing oral film dosage forms, and more particularly to the preparation of oral film dosage forms that are suitable for a high active content film dosages for both human and animal applications. This disclosure also relates to oral film dosage formulations and processes for preparing oral film dosage forms with low solubility APIs for both human and animal applications.

### BACKGROUND OF THE DISCLOSURE

It is often desirable to administer a pharmaceutical ingredient using an oral film dosage form. Oral film dosage forms have several advantages when compared with tablet and capsules. Many people have difficulty swallowing tablets and capsules, and risk choking while attempting to swallow solid oral dosage forms, but can self-administer a film dosage form without difficulty. Similarly, administering drugs through various dosage form to animals, such as companion animal often presents unique challenges, especially where dosing precision is desired. Animals may reject portion of tablets some of which often required to be split in several pieces amplifying the dosing imprecision.

While administration of a drug in an oral film dosage form can be desirable, designing an oral dosage form that provides a desirable absorption profile remains a challenge. Oral film formulations have the potential to enhance the rate of absorption of drug or active pharmaceutical ingredient (API). Despite the desired advantages of oral film formulation, suitable oral film formulations have so far had very limited applicability in part due to the limited API content of known oral films. The low API content of oral film is derived from several factor inherent to oral films such as the size limitation, whether surface wise or thickness that are in part dictated by pharmacokinetics but also by the size of the person or animal mouth. These unfortunate limitations of oral films have left the potential of oral film formulation relatively unexploited which is apparent by the limited number of approved drugs having oral film dosage form and yet no approved oral film dosage form for animal use.

These and other inefficiencies and opportunities for improvement are addressed and/or at least partially overcome by the systems, assemblies and methods of the present disclosure.

### SUMMARY OF THE DISCLOSURE

Disclosed is an oral film dosage form and process having a high active loading. The present disclosure relates to an oral film dosage having a formulation comprising an API to polymer ratio or API to total weight ratio of at least 22% preferably 30%, most preferably 37% with an API content of at least 60 mg in a film of less than 260 mg total dry weight.

According to some aspects of the disclosure, the oral dosage form delivers API which has a substantial component of enteral absorption.

According to some aspects of the disclosure, the API delivered by the oral dosage form is Maropitant.

The present disclosure relates to the use of an oral film that comprises between about 40 mg to about 80 mg of active within 4 to 9 cm².

According to some aspects of the disclosure the oral film dosage form comprises API, a suspending agent/viscosity increasing agent, a mucoadhesive film former, an amphiphilic solubility enhancer and a non-amphiphilic solubility enhancer, an agglomeration inhibitor, a surfactant, a co-surfactant, and preservative.

According to some aspects of the disclosure, the disclosed oral film dosage form comprises API, a suspending agent/viscosity increasing agent, a mucoadhesive film former, an amphiphilic solubility enhancer and a non-amphiphilic solubility enhancer, an agglomeration inhibitor, a surfactant and a co-surfactant.

According to some aspects of the disclosure the oral film dosage form comprises API, the disclosed oral film formulation comprises a suspending agent/viscosity increasing agent, a mucoadhesive film former, a solubility enhancer, an agglomeration inhibitor, a surfactant and a co-surfactant.

According to some aspects of the disclosure the oral film dosage form comprises API, the disclosed oral film formulation comprises a suspending agent/viscosity increasing agent, a mucoadhesive film former, an amphiphilic solubility enhancer and a non-amphiphilic solubility enhancer, an agglomeration inhibitor and a surfactant.

According to some aspects of the disclosure the oral film dosage form further comprises a co-surfactant.

According to some aspects of the disclosure the oral film dosage form comprises API, a suspending agent/viscosity increasing agent, a mucoadhesive film former, an amphiphilic solubility enhancer and a non-amphiphilic solubility enhancer, a surfactant and a co-surfactant.

In certain aspects of this disclosure, a formulation and process is disclosed for manufacturing a high API content oral film dosage form.

In certain aspects of this disclosure, a high API content oral film dosage form formulation is disclosed for administering.

In certain aspect of this disclosure, a process is disclosed for mitigating oral film brittleness and or API segregation associated with high content API.

In certain aspects of this disclosure, the disclosed oral film formulation comprises a suspending agent/viscosity increasing agent to API ratio between about 1:5 to about 1:15.

In certain aspects of this disclosure, the disclosed oral film formulation comprises a suspending agent/viscosity increasing agent to surfactant ratio between about 1:2.3 to about 1:6.5.

In certain aspects of this disclosure, the disclosed oral film formulation comprises a mucoadhesive film former to API ratio between about 1:0.74 to about 1:1.5.

In certain aspects of this disclosure, the disclosed oral film formulation comprises both amphiphilic solubility enhancer and non-amphiphilic solubility enhancer in a ratio of between about 9:1 to about 1:1 with a ratio of total solubility enhancer to API of between about 1:0.6 to 1:3.

In certain aspects of this disclosure, the disclosed oral film formulation comprises non-amphiphilic solubility enhancer in a ratio to API content of between about 0.03:1 to about 0.33:1.

In certain aspects of this disclosure, the disclosed oral film formulation comprises agglomeration inhibitor in a ratio to API content of between about 1:2.3 to about 1:16.

In certain aspects of this disclosure, the disclosed oral film formulation comprises surfactant in a ratio to API content of between about 1:1 to about 1:4.

In certain aspects of this disclosure, the disclosed oral film formulation comprises a co-surfactant and a surfactant in a ratio of between about 1:1 to about 1:3.25 and wherein the ratio of co-surfactant to API content of between about 1:2.2 to about 1:7.

In certain other aspects of this disclosure, the oral film for providing transport of an agent in a buccal cavity of a subject, comprises a dissolvable or disintegrable film matrix.

In certain aspects of this disclosure, the oral film matrix will have an API that is solubilized in film matrix, or pre-solubilized then dispersed in film matrix, or suspended in film matrix, embedded in polymeric matrix, or a combination thereof.

In certain aspects of this disclosure, the disclosed oral film formulation comprises a low solubility API, a surfactant, an acidic pH and at least two polymers, wherein the low solubility API is at least 22% by weight, preferably 30% by weight, most preferably 37% by weight of the oral film formulation.

In certain aspects of this disclosure, the low solubility API is Maropitant.

In certain aspects of this disclosure, the disclosed oral film formulation has a pH less than 5.

In certain aspects of this disclosure, the disclosed formulations and excipients are specifically adapted for use in animals.

In certain aspects of this disclosure, the disclosed formulations further comprise flavors and flavor enhancers to improve palatability for animals.

These and other features, advantages and objects of the various embodiments will be better understood with reference to the following specification and claims.

### BRIEF DESCRIPTION OF THE FIGURES

FIG. 1 Illustrates film 143P24 in a mixture of methyl ethyl ketone (MEK), poly ethylene glycol (PEG 300), and methanol.

FIG. 2 illustrates film 143P80A in simulated saliva pH 6,8 without shaking at 16 minutes.

FIG. 3 illustrates film 143P80B in simulated saliva pH 6,8 without shaking at 16 minutes. Similarly, FIGS. 4 and 5 illustrate film 143P80A (FIG. 4) versus 143P80B (FIG. 5) in simulated saliva pH 6,8 without shaking at 21 minutes.

FIG. 4 illustrates film 143P80A in simulated saliva pH 6,8 without shaking at 21 minutes.

FIG. 5 illustrates film 143P80B in simulated saliva pH 6,8 without shaking at 21 minutes.

FIG. 6 illustrates film 143P81A blood data in dogs.

FIG. 7 illustrates injectable Maropitant in dogs.

FIG. 8 illustrates film 143P81A compared to Maropitant injectable in dogs.

FIG. 9 is a graphical depiction of permeability results of Maropitant at different pHs.

FIG. 10 is a graphical depiction of permeability results of Maropitant films with and without citric acid.

### DETAILED DESCRIPTION

The oral film dosage form disclosed herein generally involves a film formulation comprising a high content of API or high percentage (w/w) of API to increase oral film dosage form API loading potential and potentially mitigating any known defect generally associated with oral films with such a high API content.

An “oral film dosage form” generally refers to an edible composition that can be ingested by a subject (human or animal) to orally administer a predetermined amount of an active agent(s) to the subject, wherein the composition is in the form of a film.

The term “film” refers to a type of dosage form that is distinctly different from pills, tablets, caplets, and capsules, and in which the dosage form is a thin strip of material. Such films are typically rapidly disintegrating or rapidly dissolving, but can also exhibit longer disintegration time when required. The films are generally sufficiently flexible to allow bending or even folding without breaking. The films typically have length and width dimensions on the order of 5 to 35 mm, although larger or smaller dimensions are possible and may be desirable in particular circumstances, and a thickness on the order of 5 to 300 μm, although larger or smaller thicknesses are possible and may be desirable in certain circumstances.

The term “matrix” or “film matrix” refers to the polymer component or mixture of polymers, which creates the film forming matrix supporting the API within the oral film dosage form.

The term “active agent(s)” refers mainly to active pharmaceutical ingredients (API), but may also refer generally to any agent(s) that chemically interacts with the subject to which it is administered to cause a biological change, such as, but not limited to, eliminating symptoms of disease or regulating biological functions.

The term “amorphous” refers to the non-crystalline form of the solid, a state that lacks the regular crystalline organization of atoms. The amorphous content (amorphicity) of a solid can be accurately and precisely assessed using a number of well-established methodologies, including isothermal calorimetry, Powder X-ray Diffraction (PXRD), Differential Scanning calorimetry (DSC), Continuous Relative Humidity Perfusion, Microcalorimetry (cRHp), and Dynamic Vapor Sorption (DVS). In this document, the term amorphous also refers to an active agent(s) that exhibits 30% or more than 30% of amorphous material, more preferably above 50%.

The term “stable” refers to a product which exhibits no changes in the dissolution profile or remains within the established specifications and recovery when the product is exposed to normal stability conditions (e.g., 25° C./60% RH and 40° C./75% RH) for an extended period of time while also demonstrating no chemical degradation.

The term “non-solubilized” means that the majority of the crystalline, amorphous or partially amorphous active agent(s) is uniformly distributed as solid particles into a polymer matrix (e.g., a continuous and homogenous semi-solid phase). The stability of an API can be increased in a finished film product by using the API as a partially/non-solubilized dispersion. A solubilized API, particularly a crystalline API, may re-crystallize over time, during shelf life and storage, which may adversely affect the overall bioavailability of the product. The choice of a partially/non-solubilized API can also be used to control the dissolution behavior and release of API from a film dosage for systemic uptake within a patient. Uptake and absorption of API is governed by the drug solubility and availability, thus controlling its crystallinity and particle size allows us to influence the bioavailability of the API within the human or animal body.

The mean particle size diameter (D50) equal or lower than 250 μm, refers to the size distribution of the solid particle uniformly distributed in the matrix film. The size can be small enough to avoid any roughness texture or unpleasant mouth feel experience when orally ingested.

The term “suspended” (and variations thereof) refers to a dispersion of solid material (e.g., particles or powder) in a bulk liquid medium, in which the solid material is not completely dissolved on a molecular level, and will eventually settle out of the liquid in the absence of agitation. In a suspension, the suspended material is not completely dissolved in the liquid.

The term “polymer” refers to a long molecule chain made of many repeating units.

The term “Water soluble ingredient” refers to an ingredient able to be dissolved in a particular solvent.

The term “non-water soluble ingredient” refers to ingredient meaning not able to be dissolved in a particular solvent.

The terms “suspending agent” (also referred to as a “viscosity increasing agent”) refers to water soluble ingredients or non-water soluble ingredients or combination thereof employed to prevent adjacent suspended particles from coming close enough to join each other by increase sufficiently the viscosity of the drug vehicle, and enables by steric stabilization the suspension to be stably maintained, beside above properties certain suspending agent/viscosity increasing agent additionally interact with biological mucosa to create an strengthen oral film mucoadhesion. Examples comprise polysaccharide in the form of one or a mix of Hydroxypropylmethylcellulose (HPMC) where the polymer structure combines both hydrophobic (methoxy group) and hydrophilic substitutions (hydroxypropoxy group) where the 2% aqueous viscosity is between about 1298 to about 5040 millipascal second (mPas) (2%, 20 C), Hydroxypropyl Cellulose (HPC) where the 2% aqueous viscosity is above about 150 mPas (2%, 25 C), hydroxyethyl cellulose (HEC), Gums such as water soluble carboxymethyl cellulose (CMC), Gellan, propylene glycol alginate, water soluble alginate salt, Acacia, Pectin, Xanthan, guar gum, carrageenan, and water insoluble alginates derivatives, water insoluble CMC derivatives, colloidal silicon dioxide, Agar, Locust bean, tragacanth. It also comprise Polyvinylpyrrolidone of Molecular Weight (MW) of 1 000 000 MW and above (K-value of 85 and above) with aqueous viscosity of 300 mPAs (10%, 20 C) and above and higher molecular weight polyethylene oxide (PEO) (MW above 600 000). The following are excluded from the definition of the terms “suspending agent/viscosity increasing agent”: one or a mix of HPMC where the polymer structure do not combines both hydrophobic and hydrophilic substitutions, and or having aqueous viscosity below 1298 mPas or above 5040 mPas (2%, 20 C), Methyl cellulose (MC), Microcrystalline cellulose (MCC), powdered cellulose, Sodium Starch Glycolate, starch, Polyvinylpyrrolidone of MW below 1.000.000 MW and K-value below 85 and with aqueous viscosity of less than 300 mPAs (10%, 20 C), polyvinylpyrrolidone-vinyl acetate copolymer, polyplasdone crospovidone, HPC where the 2% aqueous viscosity is below 150 mPas (2%, 25 C), water insoluble bentonite.

The terms “surfactant” refers to surfactant(s) of an Hydrophilic Lipophilic Balance (HLB) of 7 and higher having an amphiphilic structure, with polar hydrophilic head (ionic or no-ionic) and non-polar hydrophobic tail. Surfactants are employed to dissipate the free surface energy of particles by reducing the interfacial tension and contact angle between the solid and the suspending vehicle and comprise PEG 300 oleic glycerides (Labrafil® M-1944CS), PEG 300 linoleic glycerides (Labrafil® m-2125CS); Hydroxylated lecithin; Caprylocaproyl polyoxyl-8 glycerides; Polyoxyethylene (4) sorbitan monostearate, Polyoxyethylene 20 sorbitan tristearate, Polyoxyethylene (5) sorbitan monooleate, Polyoxyethylene 20 sorbitan trioleate; Sorbitan Esters (Sorbitan Fatty Acid Esters) such as: Sorbitan monolaurate, Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene 20 sorbitan monooleate, Polyoxyethylene 20 sorbitan monoisostearate Polyethylene glycol monostearate (Gelucire 48/16), poloxamer having MW up to 14.600, viscosity up to 3100 mPAs (77 C) but exclude surfactant(s) of an HLB below 7 such as Propylene glycol monocaprylate type I, Propylene glycol monocaprylate type II, Propylene glycol monolaurate, Sorbitan monoisostearate, Sorbitan monooleate, Sorbitan monopalmitate, Sorbitan monostearate, Sorbitan sesquioleate, Sorbitan trioleate, Sorbitan tristearate, glyceryl monoleate.

Though not always required, a co-surfactant acts as a second surfactant and can help drug solubilization. Co-surfactants pair with a surfactant to lower interfacial tension sufficiently between the solid and suspending vehicle. Co-Surfactants comprise Short- and medium-chain alcohols, such as ethanol, isopropanol, or propylene glycol; Polyethylene Glycols such PEG 400, alkanetriol such as glycerol; N-methyl pyrrolidone (Pharmasolve), 2-Pyrrolidone (Soluphor P); Diethylene glycol monoethyl ether (Transcutol™), Propylene glycol monocaprylate type I, Propylene glycol monocaprylatetype II, Polyoxyethylene 20 sorbitan monolaurate, Polyoxyethylene 20 sorbitan monooleate but exclude pentanediol, hexanediol, octanediol. Co-surfactants can act at times as solubility enhancers and in certain case as plasticizer.

The term “solubility enhancer” also refers to excipients used to solubilize low solubility drugs via non-covalent interactions, and permit dissolution and bioavailability enhancement of the said drug. Non-covalent interactions include van der Waals forces, hydrogen bonding, dipole-dipole and ion-dipole interactions, and in certain cases favorable electromagnetic interactions. In this disclosure, the solubility enhancer involves the combination of two types of excipients: type (I) of Amphiphilic structure having both hydrophobic and hydrophilic constituents and type (II) of non-amphiphilic structure having either majority of hydrophilic constituents or majority of hydrophobic constituents or combination thereof. Solubility enhancers are divided in two categories, amphiphilic solubility enhancers A) Cellulosic of low Molecular Weight (MW) Hydroxypropyl Cellulose (HPC) (up to 95.000) having aqueous viscosity of Not More Than (NMT) 150 mPas (5%, 25 C), B) Surfactant(s) of an HLB from 3 to 12 (category I) or of an HLB equal to 12 and above (category II) or a combination of the two categories: sodium lauryl sulfate, copolymers of poly(ethylene oxide) (PEO) and poly(propylene oxide) (PPO) i.e poloxamer having MW up to 14.600, viscosity up to 3100 mPas (77 C); PEG 300 oleic glycerides, PEG 300 linoleic glycerides; Sorbitan Esters (Sorbitan Fatty Acid Esters) such as: Sorbitan monoisostearate, Sorbitan monolaurate, Sorbitan monooleate, Sorbitan monopalmitate, Sorbitan monostearate, Sorbitan sesquioleate; Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene (4) sorbitan monostearate, Polyoxyethylene 20 sorbitan tristearate, Polyoxyethylene 20 sorbitan monooleate, Polyoxyethylene (5) sorbitan monooleate, Polyoxyethylene 20 sorbitan trioleate, Polyoxyethylene 20 sorbitan monoisostearate Propylene glycol monocaprylate type I and type II, Caprylocaproyl polyoxyl-8 glycerides. C) Polyvinylpyrrolidone of up to 1.500.000 MW, having aqueous viscosity of NMT 700 mPAs (10%, 20 C) D) polyethylene oxide (PEO), of up to 200.000 MW, having aqueous viscosity of NMT 90 mPAs (5%, 25 C). Cyclodextrines. The second category of Non-Amphiphilic solubility enhancers type (II): A) With majority of hydrophilic constituents: Glycerol, Propylene glycol, and PEGs. Said PEGs of up to 6600 MW and of viscosity of Not More Than (NMT) 390 mPas (at 98.98 C+/−0.3 C) B) With majority of hydrophobic constituents such as oily surfactant of Hydrophilic Lipophilic Balance (HLB) below 3 and oily solubility enhancer: Medium chain triglycerides (MCT) and Glycerol monolinoleate (Maisin CC™), soybean oil, Olive oil, Sorbitan trioleate, Sorbitan tristearate.

The terms “Mucoadhesive film former” refers to polymers that form the film matrix, film strip, film sheet and dissolves in aqueous environment and gives bio-adhesive properties to the mucosa examples comprising PEO, Pullulan, CMC, HPC, HPMC and exclude ethyl cellulose (EC), polyvinyl alcohol (PVA), Starch, Polymethacrylate polymers.

The terms “Agglomeration inhibitor” refers to hydrophilic polymer used to prevent micro-size poorly soluble drug particles that tend to grow with time, and favored for them a more thermodynamically stable distribution comprising Polyvinylpyrrolidone and Hydroxypropylmethylcellulose (HPMC) where the polymer structure combines both hydrophobic (methoxy group) and hydrophilic substitutions (hydroxypropoxy group) and have aqueous viscosity up to 5040 mPas (2%, 20 C), employed alone or mixed with Methyl cellulose (MC) of aqueous viscosity up to 5040 mPas (2%, 20 C) and exclude HPMC of aqueous viscosity superior to 5040 mPas (2%, 20 C) and MC alone.

The term “Preservative agent” refers to ingredient that is pharmaceutically acceptable to kill any bacteria and prevent mold growth that may result during drug storage examples comprising methyl propylparaben, propylparaben, benzalkonium chloride, propylene glycol, and benzoic acid.

A film layer is a sheet-like material having a thickness that is much less than its length or width. For example, oral transmucosal devices typically have a thickness on the order of about 50 μm to 500 μm (i.e., 0.05 mm to 0.5 mm), although thicker or thinner films may be suitable; and width and length dimensions typically on the order of about 5 mm to 36 mm, though larger or smaller dimensions can be used. The film layer could as well be round and oval in shape, and have straight or rounded corners. Film layer according to the present disclosure is understood as an active containing film layer that is equals to or thinner than 1.5 mm.

The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication or simply against constant washing of the saliva. This protective layer also serves to favor the directed absorption of the API within the oral cavity rather than enteric uptake in the gastrointestinal (GI) tract. The term “mucoadhesive or bioadhesive” means that the composition of the film layer is formulated to adhere to the mucous membrane through which delivery of the active agent is targeted. For example, bioadhesive polymers used in formulating the film should be selected to exhibit adequate adhesion within the environment at the targeted mucous membrane to ensure that the bioadhesive layer remains in contact with the mucous membrane to which it is applied and allows the active agent to directly enter the blood stream through the mucous membrane. Mucoadhesive ingredients are often responsible for the strong bond between the product and the mucosal surface.

The term “Flavoring Agents or Flavor” and variations thereof generally refers to concentrated preparations, with or without flavor adjuncts required in their manufacture, used to impart flavor, with the exception of salt, sweet, or acid tastes. Flavoring agents may be classified as natural, artificial, or natural and artificial (N&A) by combining the all natural and synthetic flavors or other forms known in the art. Flavouring agents are categorized by their physical classification as solid flavoring agents and liquid flavoring agent.

The term “Flavor Enhancer” and variations thereof generally refers to compounds that particularly enhance certain tastes or reduce undesirable flavors without having an especially strong taste of their own. They harmonize taste components and make food/drug preparations more palatable. Examples include but are not limited to maltol, ethyl maltol and monosodium glutamate, glutamic acid, glutamates, purine-5-ribonucleotides, inosine, guanosine, adenosine 5-monophosphates, sugars, sweetener, carboxylic acids (e.g., citric, malic, and tartaric), common salt (NaCl), amino acids, some amino acid derivatives (e.g., monosodium glutamate-MSG), and spices (e.g., peppers) are most often employed, yeast, yeast extract, dried yeast and others or mixtures thereof.

The term “Sweetener” and variations thereof generally refers to a solid or liquid ingredient that is used to impart a sweet taste to food or drug product. Sweeteners are often classified as either nutritive (caloric) or non-nutritive (non-caloric), natural or synthetic. Examples of Sweetener include but are not limited to sucrose, dextrose, lactose, glucose, advantame, sorbitol, mannitol, liquid glucose, honey molasses, saccharin, sucralose, rebaudioside A stevia, rebaudioside M stevia, stevioside, mogroside IV, mogroside V, alitame, saccharin, neohesperidin dihydrochalcone, cyclamate, neotame, N-[3_(3-hydroxy-4-methoxybenzyl yl) propyl]-L-α-aspartyl]-L-phenylalanine 1-methyl ester, N-[3-(3-hydroxy-4-methoxyphenyl)-3-methylbutan yl]-L-α-aspartyl]-L-phenylalanine 1-methyl ester, N-[3-(3-methoxy-4-hydroxyphenyl) propyl]-L-α-aspartyl]-L-phenylalanine 1-methyl ester, curculin, cyclamate, aspartame, acesulfame potassium and others or mixtures thereof.

The term “plasticizer” refers to a component that reduces the glass-transition temperature of the film forming polymers (e.g., the water soluble polymer or water soluble polymers in the film). The plasticizer increases the flexibility, enhances elasticity and reduces brittleness of the film. Examples of plasticizers that can be used in the disclosed film oral dosage forms include triacetin, triethyl citrate, tributyl citrate, acetyl tributyl citrate, acetyl triethyl citrate, trioctyl citrate, acetyl trioctyl citrate, trihexyl citrate, dibutyl sebacate, PEG 300, PEG 400, Glycerine, etc. Plasticizer may be added in an amount up to 25% of the total mass of the film oral dosage form, such as from 0.5% to 25%, 1% to 20%, 2% to 15% or 5% to 10%.

The poor water solubility of the poorly water-soluble substance to be used for the solid preparation of the present disclosure means the property associated with difficulty of solubilizing the API in water. In the present disclosure, for example, the solubility of a poorly water-soluble substance at 37° ° C. is not more than 10 mg/L, preferably not more than 1 mg/L, more preferably not more than 0.5 mg/L.

An API or drug product is considered highly soluble when the highest dose strength is soluble in <250 ml water over a pH range of 1 to 7.5 as per the Biopharmaceutics Classification System (BCS).

As used herein, the solubility is determined as follows. First, an excess amount of a poorly water-soluble substance is added to purified water (5 ml). The obtained mixture is incubated in a thermostatic tank at 37° C. for 30 min, and stirred in a voltex mixer. The cycle of incubating and stirring is repeated 3 more times, and the obtained suspension is filtered through a syringe filter (manufactured by Japan Pall, trade name: Acrodisc LC25, PVDF, pore size 0.2 mm). The concentration (mg/L) of the poorly water-soluble substance in the filtrate is taken as the solubility.

According to certain embodiments, the oral film formulation comprises a film matrix and an API. Maropitant was used herein as an exemplary active for demonstrating the applicability of the present disclosure. The API was initially fully solubilized in the solvent system, a mixture of methyl ethyl ketone (MEK), poly ethylene glycol (PEG 300), and methanol. Referring now to Table 1, the API was formulated in a film formulation comprising a solvent system. Solvent systems present additional challenges such as choice of film former polymers, since the list of film former polymers that are soluble in organic solvent and water is limited when compared to the large quantity of available water soluble film forming polymers. This limited choice of compatible polymers to address the formulation represent a significant hurdle to overcome when developing a solvent based oral film formulation with a polymeric film matrix. In the presently illustrated formulation (Table 1), the formulation 143P24A wet blend was coated on a liner before being dried at a temperature of 40° C. for a 60 minutes period, resulting a coated web of films with API content of 60 mg per film surface area of 20×30 mm. Those oral films showed good film forming properties, however recrystallization of the API was observed (see FIG. 1) short after manufacturing. Recrystallization of the API is undesired in oral film development because it may affects oral film critical properties such as dissolution profile, API stability, mechanical film properties.

TABLE 1
Formulation 1

| | Formulation | Weight | % Wet | % Dry | Weight (mg) |
|---|---|---|---|---|---|
| Order | 143P24A | (g) | (wt/wt) | (wt/wt) | per Film |
| 4 | Maropitant | 1.50 | 8.50 | 30.94 | 60.00 |
| 1 | PEG 300 | 0.20 | 1.13 | 4.13 | 8.00 |
| 6 | PVP K90 | 2.31 | 13.08 | 47.60 | 92.32 |
| 7 | HPC LF | 0.77 | 4.34 | 15.78 | 30.60 |
| 5 | HPC GXF | 0.08 | 0.43 | 1.55 | 3.01 |
| 3 | MEK | 10.37 | 58.75 | — | — |
| 2 | Methanol | 2.43 | 13.78 | — | — |

| | | | | |
|---|---|---|---|---|
| TOTAL | 17.65 | 100.0 | 100.0 | 193.93 |

To mitigate the apparent crystallization obtained in previously illustrated Formulation 1, an alternate formulation and an alternate process were used. The API was instead suspended in particulate form in the aqueous solution of the polymer used for casting the film. Using a suspended API approach help preventing or at least mitigating uncontrolled undesired recrystallization in the cast oral film. Using the particulate API in aqueous solution with the polymer results in a uniformly distributed biphasic films, containing suspended API therein. This alternate formulation and process, though adequate at reducing, mitigating and/or preventing recrystallization presents additional significant challenges regarding API content uniformity per oral film dosage. It also presents significant challenges regarding oral film texture and often yield unacceptable film mechanical properties. Oral film mechanical properties may be unacceptable for several reasons, such as having an oral film product that is brittle, thus cracks over time, a product that is not stiff enough, a product that does not have the adequate adhesion properties to allow suitable packaging of the cast oral film dosage form. Referring now to Table 3, the API was formulated as a suspension in methanol using Maropitant (active ingredient Biopharmaceutics Classification System, BCS class II). This formulation resulted in the formation of large crystals in the methanol before the addition of film former polymers, representing a formulation failure. The addition of portion of water to the previous solution resulted in big aggregates.

TABLE 2
Formulation 2

| | Formulation | Weight | % Wet | % Dry | Weight (mg) |
|---|---|---|---|---|---|
| Order | 143P30 | (g) | (wt/wt) | (wt/wt) | per Film |
| 1 | Labrafil 2125 CS | 0.5 | 2.86 | 10.02 | 30.30 |
| 2 | Tween 20 | 0.5 | 2.86 | 10.02 | 30.30 |
| 3 | Methanol | 10 | 57.18 | — | — |
| 4 | Maropitant | 0.99 | 5.66 | 19.84 | 60.00 |
| 5 | Purified water | 2.50 | 14.29 | — | — |
| N/A | Film formers | 3 | 17.15 | 60.12 | 181.82 |

| | | | | |
|---|---|---|---|---|
| TOTAL | 17.49 | 100 | 100 | 100 |

Following the failure of Formulation 2, the API was incorporated as dispersed particles in a novel formulation. This novel formulation, Formulation 3 (refer to Table 3), produced in an oral film exhibiting an undesirably high level of stickiness to the support liner. Proper adhesion properties are important in oral film development when assessing the ability to scale up from the laboratory scale formulation to a commercial scale process. This undesirably high adhesion renders the film formulation unsuitable for large scale manufacturing. In addition, Formulation 3 exhibits longer disintegration time. The disintegration time for this Formulation 3 (Refer to Table 3) is 6 fold longer when compared to the similar formulation using a different API (referring to Formulation 4, Table 4), namely Tadalafil, and that despite the fact that Tadalafil is from the same BCS class II as maropitant and present in the same amount in the formulation. Therefore demonstrating a surprisingly significant difference in disintegration properties of seemingly similar API when used in a high active concentration or high loading oral film. This is significant because the higher the amount of active in the oral film (higher active concentration) the more the film properties will be affected by such active physical and chemical properties, most likely its physical ability to interact in a stable manner with the oral film matrix.

TABLE 3
Formulation 3

| | Formulation | Weight | % Wet | % Dry | Weight (mg) |
|---|---|---|---|---|---|
| Order | 143P37A | (g) | (wt/wt) | (wt/wt) | per Film |
| 5 | Maropitant | 4.00 | 8.69 | 36.26 | 60.00 |
| 1 | Labrafil 2125CS | 1.00 | 2.17 | 9.07 | 15.00 |
| 2 | Tween 20 | 1.00 | 2.17 | 9.07 | 15.00 |
| 4 | water | 35.00 | 76.04 | — | — |
| 3 | Glycerine | 0.62 | 1.35 | 5.62 | 9.30 |
| 7 | PGA LV | 0.65 | 1.41 | 5.89 | 9.75 |
| 8 | Pullulan | 3.06 | 6.65 | 27.74 | 45.90 |
| 6 | Maltitol | 0.70 | 1.52 | 6.35 | 10.50 |

| | | | | |
|---|---|---|---|---|
| TOTAL | 46.03 | 100 | 100 | 165.45 |

TABLE 4
Formulation 4

| | Formulation | Weight | % Wet | % Dry | Weight (mg) |
|---|---|---|---|---|---|
| Order | 143P39D | (g) | (wt/wt) | (wt/wt) | per Film |
| 5 | Tadalafil | 4.00 | 8.69 | 36.26 | 60.00 |
| 1 | Labrafil 2125CS | 1.00 | 2.17 | 9.07 | 15.00 |
| 2 | Tween 20 | 1.00 | 2.17 | 9.07 | 15.00 |
| 4 | water | 35.00 | 76.04 | — | — |
| 3 | Glycerine | 0.62 | 1.35 | 5.62 | 9.30 |
| 7 | PGA LV | 0.65 | 1.41 | 5.89 | 9.75 |
| 8 | Pullulan | 3.06 | 6.65 | 27.74 | 45.90 |
| 6 | Maltitol | 0.70 | 1.52 | 6.35 | 10.50 |

| | | | | |
|---|---|---|---|---|
| TOTAL | 46.03 | 100 | 100 | 165.45 |

According to embodiments, Maropitant, a poorly water soluble and highly lipophilic drug (Biopharmaceutics Classification System, BCS class II), was incorporated into different OF matrices at high loading (at least 50 mg per oral film dosage form having a maximum surface of 600 mm² or having a minimum concentration of about 0.08 mg per mm²). Maropitant was used as model drug for this study. Maropitant citrate, an antiemetic drug, is given orally in tablet form or is injected under the skin to treat vomiting and motion sickness in companion animal such as dogs and cats. Despite our previous unsuccessful attempts and formulation failures, Maropitant was successfully incorporated in a high loading oral film (refer to Formulation 5, Table 5). Formulation 5 which represents an exemplary a high drug loading OF formulation comprises API, a film forming polymer, a plasticizer, co-surfactant.

According to embodiments, high drug loading oral film dosage form suitable film former polymers include but are not limited to hydroxypropyl cellulose, polyvinylpyrrolidone, propylene glycol alginate, polyethylene oxide, pullulan, and hydroxypropyl methylcellulose, gums. Suitable surfactants include but are not limited to PEG 300, (Dipolyhydroxystearate.) Suitable plasticizers/co-surfactant includes but are not limited to hydroxylated lecithin, labrafil (poly-oxyl-6 glycerides with various tails), and tween 20. In addition, high loading oral film may contain oils. High loading oral film suitable oils includes but are not limited to MCT, Maisine (glycerol monollinoleate) and flavor oils such as peppermint oil when such high flavor coverage is required. The interplay between types and amounts of the polymers, plasticizers, surfactants, and oils plays an important role on mechanical strength as well as dissolution, disintegration, and mucoadhesion performance of the high loading OFs.

The issues arising during the development of a high drug load OF are mainly related to its mechanical properties and mucoadhesion. Therefore, the impact of API loading, the concentration of the surfactants, plasticizers and/or polymers on flexibility, disintegration time, folding and stickiness were preliminarily evaluated.

According to embodiments, the film blend uses water based, solvent based or a combination of solvent- and water-based systems. The API in certain aspects of the present disclosure is loaded as dispersed in film matrix, or solubilized in film matrix, or pre-solubilized then dispersed in film matrix, embedded in polymeric matrix, or combination thereof. Different film formulations were prepared by varying combination and concentration of polymers, surfactants, and plasticizers in different solvent systems.

Disclosed herein are three different formulation approaches, suspended, dissolved and pre-solubilized.

The suspended API based oral film formulation is prepared by suspending API in liquid blend which is then being dispersed within the solid oral film matrix during following coating and drying. (examples: 143P81B, 143P37A).

The film blend manufacturing process in this exemplary formulation involves a water based system where the API is suspended in an amphipathic surfactant such as but not limited to Hydroxylated Lecithin and co-surfactant such as but not limited to Glycerine which is then homogenized. Once the homogenization step has been conducted the API suspension is stabilized with the addition of high viscosity polymers such as but not limited to HPMC (E4M). yet, before the introduction of the film forming polymers, the plasticizer such as but not limited to (PEG 300) is introduced to the blend. The plasticizer is added to optimize oral film flexibility in finished final dry OF product. Once the plasticizer has been added, a low viscosity polymer such as but not limited to HPC (SSL) is added to the previous wet blend. The low viscosity polymer is thus blended together with the previous API suspension to assist incorporation of the organic API in the aqueous system. The addition of the low viscosity polymer is followed by the addition of the HPMC E50 to prevent API crystal growth in the final dry OF. To flavor the wet blend, the flavor (i.e. Natural Pet Flavor (PC-0050)), flavor enhancer (i.e. Savorboost BK), and sweetener (i.e. Magnasweet) are blended with the wet blend to create the acceptable flavor profile in the final dry OF. At the last step of the blending process, the main film former polymer such as but not limited to PEO is added and mixed with the blend. The final blend is then deaerated to remove the presence of air bubbles. Upon completion of the deaeration process, the blend is poured on the flat liner at specified gap, the coated liner is then oven dried to generate the oral film web. The oral film web is then cut into desired dimension, and packaged in individual pouches.

TABLE 5
Formulation 5

| | | | | | | Weight |
|---|---|---|---|---|---|---|
| | Formulation | | Weight | % Wet | % Dry | (mg) |
| Order | 143P81B | Function | (g) | (wt/wt) | (wt/wt) | per Film |
| 4 | API | Responsible for therapeutic | 1.50 | 8.11 | 27.30 | 60.00 |
| | | effect | | | | |
| 3 | Hydroxylated | surfactant | 0.50 | 2.70 | 9.10 | 20.00 |
| | Lecithin | | | | | |
| 2 | Water | Solvent system | 13.00 | 70.29 | — | — |
| 1 | Glycerine | Co-surfactant/plasticizer | 0.24 | 1.27 | 4.28 | 9.40 |
| 5 | HPMC E4M | Suspending agent/viscosity | 0.15 | 0.81 | 2.73 | 6.00 |
| | | increasing agent | | | | |
| 7 | HPC SSL | Amphiphilic solubility | 0.33 | 1.78 | 6.01 | 13.20 |
| | | enhancer | | | | |
| 6 | PEG 300 | Co-surfactant/plasticizer | 0.20 | 1.08 | 3.64 | 8.00 |
| 9 | HPMC E50 | Agglomeration inhibitor and | 0.33 | 1.78 | 6.01 | 13.20 |
| | | mucoadhesive film former | | | | |
| 10 | PEO 200K | Water soluble mucoadhesive | 1.70 | 9.19 | 30.94 | 68.00 |
| | | film former | | | | |
| 8 | Savorboost | Flavor enhancer | 0.05 | 0.27 | 0.91 | 2.00 |
| | BK | | | | | |
| 8 | Natural Pet | Flavor | 0.30 | 1.62 | 5.46 | 12.00 |
| | Flavor (PC- | | | | | |
| | 0050) | | | | | |
| 8 | Magnasweet | Sweetner | 0.20 | 1.08 | 3.64 | 8.00 |

| | | | | |
|---|---|---|---|---|
| TOTAL | 18.50 | 100.0 | 100.0 | 219.80 |

According to other embodiments, referring now to Formulation 6, Table 6, using the similar film blend manufacturing process, the API is homogenized to create a suspension in water with two surfactants of different solubility (i.e. water dispersible surfactant Labrafil 2125 CS (Linoleoyl Polyoxyl-6 glycerides) and water soluble surfactant Tween 20) and a plasticizer (i.e. Glycerine). Later after first homogenization, the API suspension is stabilized with the assistance of high viscosity polymer such as but not limited to PGA LV (Propylene Glycol alginate Low Viscosity). Finally, the main film forming polymers (i.e. Pullulan) is blended with API suspension. After blend deaeration, the blend follows the same process as described previously to generate the finish OFs packaged in individual pouches.

TABLE 6
Formulation 6

| | Formulation | Weight | % Wet | % Dry | Weight (mg) |
|---|---|---|---|---|---|
| Order | 143P37A | (g) | (wt/wt) | (wt/wt) | per Film |
| 5 | API | 4.00 | 9.49 | 36.25 | 60.00 |
| 1 | Labrafil 2125Cs | 1.00 | 2.37 | 9.06 | 15.00 |
| 2 | Tween 20 | 100 | 2.37 | 9.06 | 15.00 |
| 4 | Water | 35.00 | 73.83 | — | — |
| 3 | Glycerine | 0.62 | 1.48 | 5.64 | 9.34 |
| 6 | PGA LV | 0.65 | 1.55 | 5.92 | 9.81 |
| 7 | Pullulan | 3.06 | 7.25 | 27.72 | 45.88 |

| | | | | |
|---|---|---|---|---|
| TOTAL | 46.04 | 100.0 | 100.0 | 165.53 |

According to some embodiments, referring now to exemplary Formulation 7, Table 7, the second approach of designing a high loading oral film is utilized, the use of pre-solubilized API within the oral film dosage form. In this process the preparation of the oral film dosage form includes solubilized API in liquid blend that will be further suspended by changing the solvation properties of the solvent system. The precipitated API in solvent is then dispersed further within the solid film matrix by coating and drying a final oral film product. (Examples: 143P26 C).

In this example, Maropitant the selected API candidate is solubilized in solvent based system made of MEK and Methanol with assistance of two surfactants (i.e. Labrafil 2125Cs and Tween 20). After API dissolution is achieved, sodium Starch Glycolate which is insoluble in the mixture MEK-Methanol is added step wise to the system to modulate API solubility in the solvent mixture. The API then precipitate as a result of the addition of the Starch Glycolate. The precipitated (or pre-solubilized) API is then dispersed in the network of the Sodium Starch Glycolate. The thickener polymer such as but not limited to HPC GXF is added to the blend support the dispersion of the API and to prevent further crystal growth. At the later stage, the main film forming polymers such as but not limited to HPC LF and PVP K90 are blended together with the wet blend to create an optimal flexibility and to prevent API crystal grow (PVP K90) in the final dry OF. The final blend is deaerated to remove the bubbles, then poured on the flat liner at specified gap, the coated liner is dried in the oven to generate film web that will be cut into desired dimension, and packaged in individual pouches.

TABLE 7
Formulation 7

| | Formulation | Weight | % Wet | % Dry | Weight (mg) |
|---|---|---|---|---|---|
| Order | 143P26C | (g) | (wt/wt) | (wt/wt) | per Film |
| 4 | API | 2.00 | 10.10 | 28.57 | 55.41 |
| 1 | Labrafil 2125Cs | 0.50 | 2.53 | 7.14 | 13.85 |
| 1 | Tween 20 | 0.50 | 2.53 | 7.14 | 13.85 |
| 7 | PVP K90 | 2.00 | 10.10 | 28.57 | 55.41 |
| 7 | HPC LF | 0.75 | 3.79 | 10.71 | 20.78 |
| 6 | HPC GXF | 0.25 | 1.26 | 3.57 | 6.93 |
| 5 | Na Starch | 1.00 | 5.05 | 14.29 | 27.70 |
| | Glycolate | | | | |
| 3 | MEK | 10.37 | 52.36 | — | — |
| 2 | Methanol | 2.43 | 12.28 | — | — |

| | | | | |
|---|---|---|---|---|
| TOTAL | 19.80 | 100.0 | 100.0 | 193.93 |

According to embodiments, Referring now to Formulation 8, Table 8, the API candidate is solubilized in solvent based system made of MEK with assistance of two surfactants (i.e. Labrafil 2125Cs and Tween 20). After API solubilisation, purified water is added stepwise to the system to shift API solubility and to control the API particle sizes growth to obtain a fine suspension. Water soluble film former polymers and thickener are blended together with the wet blend to create stable dispersed suspension system. The final blend is deaerated to remove the bubbles, then poured on the flat liner at specified gap, the coated liner is dried in the oven to generate film web that will be cut into desired dimension, and packaged in individual pouches.

TABLE 8
Formulation 8

| | Formulation | Weight | % Wet | % Dry | Weight (mg) |
|---|---|---|---|---|---|
| Order | 143P32 | (g) | (wt/wt) | (wt/wt) | per Film |
| 1 | Labrafil 2125 CS | 0.5 | 2.02 | 8.33 | 15.00 |
| 2 | Tween 20 | 0.5 | 2.02 | 8.33 | 15.00 |
| 3 | MEK | 15 | 60.61 | — | — |
| 4 | Maropitant | 2 | 8.08 | 33.33 | 60.00 |
| 5 | Purified water | 3.75 | 15.15 | — | — |
| 6 | Film formers | 3 | 12.12 | 50.00 | 90.00 |

| | | | | |
|---|---|---|---|---|
| TOTAL | 24.75 | 100 | 100 | 180.00 |

According to some embodiments, the API is solubilized in the oral film dosage form. In this process the preparation of the oral film dosage the API is solubilized in the liquid blend with dissolved carrier excipients. Once the API is solubilized with the dissolved carrier excipient this solution gets dispersed within the solid film matrix by upon coating and drying. (Examples: 143p23A, 143p23B)

Referring now to Formulation 9, Table 9, selected API candidate, Maropitant, is solubilized in solvent based system made of MEK and methanol with assistance of an oil vehicle MCT and a co-surfactant PEG 300. After API dissolution, water soluble film former polymers Co-povidone and HPC LF are blended together with the wet blend to create transparent Maropitant solution. The final blend is deaerated to remove the bubbles, then poured on the flat liner at specified gap, the coated liner is dried in the oven to generate film web that will be cut into desired dimension, and packaged in individual pouches. Formulation 9 is an exemplary formulation of a suitable solvent based formulation where the API is dissolved with in the oral film matrix, thereby improving oral film content uniformity.

TABLE 9
Formulation 9

| | Formulation | Weight | % Wet | % Dry | Weight (mg) |
|---|---|---|---|---|---|
| Order | 143P23A | (g) | (wt/wt) | (wt/wt) | per Film |
| 5 | API | 4.40 | 12.03 | 39.70 | 60.00 |
| 2 | MCT | 1.62 | 4.43 | 14.63 | 22.11 |
| 1 | PEG 300 | 1.02 | 2.79 | 9.20 | 13.91 |
| 6 | Copovidone | 0.32 | 0.86 | 2.83 | 4.28 |
| 6 | HPC LF | 3.72 | 10.19 | 33.64 | 50.85 |
| 4 | MEK | 20.74 | 56.68 | — | — |
| 3 | Methanol | 4.76 | 13.02 | — | — |

| | | | | |
|---|---|---|---|---|
| TOTAL | 36.58 | 100.0 | 100.0 | 151.15 |

Referring now to Formulation 10, Table 10, is disclosed an alternative formulation of the Formulation 9 wherein the MCT oil was removed.

TABLE 10
Formulation 10

| | | | | | | Weight |
|---|---|---|---|---|---|---|
| | Formulation | | Weight | % Wet | % Dry | (mg) |
| Order | 143P23B | Function | (g) | (wt/wt) | (wt/wt) | per Film |
| 4 | API | Responsible for | 2.40 | 7.28 | 32.16 | 60.00 |
| | | therapeutic effect | | | | |
| 1 | PEG 300 | Co- | 1.02 | 3.09 | 13.67 | 25.50 |
| | | surfactant/plasticizer | | | | |
| 5 | Copovidone | Agglomeration | 0.32 | 0.95 | 4.20 | 7.84 |
| | | inhibitor and | | | | |
| | | mucoadhesive film | | | | |
| | | former | | | | |
| 5 | HPC LF | Water soluble | 3.72 | 11.31 | 49.97 | 93.22 |
| | | mucoadhesive film | | | | |
| | | former | | | | |
| 3 | MEK | Solvent system | 20.74 | 62.91 | — | — |
| 2 | Methanol | Solvent system/Co - | 4.76 | 14.45 | — | — |
| | | surfactant | | | | |

| | | | | |
|---|---|---|---|---|
| TOTAL | 32.96 | 100.0 | 100.0 | 186.55 |

Referring now to Table 11, it is disclosed that the film blend uses water based systems, solvent based systems or combination thereof. The API in certain aspect of the present disclosure is loaded as dispersed in the film matrix, solubilized in the film matrix, or pre-solubilized then dispersed in the film matrix, embedded in the polymeric matrix, or combination thereof. Different film formulations were prepared by varying concentration of polymers, surfactants, and plasticizers in different solvent systems to provide suitable oral film dosage form and optimize certain aspect of the present disclosure.

TABLE 11
Comparison between the three developed approaches

| Comparison | Approaches | | |
|---|---|---|---|
| Aspects | Suspended | Pre-solubilized | Solubilized |
| Surface area | Increased surface area | Increased surface area | Highest dispersion. |
| | that facilitates the drug | that facilitates the drug | May provide the |
| | dispersibility and its | dispersibility and its | quickest onset of |
| | dissolution compared to | dissolution compared | absorption, better |
| | physical mixtures and | to physical mixtures | improvement of |
| | pure API. | and pure API. Have | bioavailability, and |
| | | better chance for quick | allow to reduce the |
| | | onset of absorption | dosage strength |
| | | since the dispersion | compare to suspended |
| | | come from molecular | approach. |
| | | solution. | |
| Bioavailability | Will exhibited superior | Will exhibited superior | Will exhibited the best |
| | performance for | performance for | performance for |
| | enhancement of | enhancement of | enhancement of |
| | dissolution compared to | dissolution compared | dissolution. |
| | crystalline form. | to the suspended | |
| | | approach. | |
| Stability | Reduced risk of re- | Reduced risk of re- | Insufficient physical |
| | crystallization, and | crystallization, and | stability. |
| | crystallization growth. | crystallization growth. | Require more |
| | Other physical and | Other physical and | experiments and |
| | chemical stability are to | chemical stability are to | strategies to explore |
| | be investigated. | be investigated. | the way to fix it. |
| | | | Bioavailability will be at |
| | | | risk. |
| | | | Other physical and |
| | | | chemical stability are to |
| | | | investigate. |
| Formulation | Large choices of | Limited choice of | Limited choice of |
| optimization | ingredients to allow | ingredients to allow | ingredients to allow |
| | optimization. | optimization. | optimization. |
| Coating pass | Require only one | May require double | Double coating |
| (drying) | | coating | |
| Development | Lead approach under | Early development | On hold, |
| status | development; require | | recrystallization issues |
| | more experiments to | | |
| | match the all requested | | |
| | aspects of the project. | | |

Still referring to Table 11, it can be concluded that the fully solubilized approach is unsuccessful due to constant and quick recrystallization, the Pre-solubilized approach was evaluated but would necessitate more experimentation to move for be ready for scale up, the suspended approach requires limited formulation to be ready for scale up.

Numerous formulation strategies have been investigated to overcome the poor aqueous solubility of Maropitant drug substance and load a high amount of this API in the oral film. Successful approaches are achieved for formulation when the API is dispersed in film matrix, or when the API is solubilized. Other aspect of the formulation such as film matrix physical properties, disintegration rate, drug substance effective surface area and solubility were also considered to improve the film dissolution rate and thus the API bioavailability. Maropitant is a BCS class II compound, meaning that it has a low solubility and high permeability, making its aqueous solubility a limiting factor of bioavailability. A critical parameter of the developed formulation is therefore solubility in the chosen manufacturing solvent system

The following solubility studies were performed with Maropitant drug substance in various solvents and solubility enhancers. The results have revealed promising prototypes loading up to 80 mg of API in the film matrix. During solubility studies, the API was solubilized stepwise, in a series of permitted solvent (or a combination of solvent) with and without solubility enhancer. The solvent system was based on methanol, ethanol, isopropyl alcohol (IPA), acetone, methyl ethyl ketone (MEK), and water. Solubility enhancers included Maiscine CC (MGO) (HLB 1), medium chain triglyceride (MCT) oil (HLB 1), Labrafil 1944 cs (HLB 9), Labrafil 2125CS (HLB 9), Glycerine, Tween 20 (HLB 16,7), PEG 300, Propylene glycol, SLS (HLB 40). The resulting mixture was visually inspected to evaluate solubilization. The main equipment used were glass bottles, magnetic stir bars, stirrer plate, and sonicator. The results are shown below in Table 12.

TABLE 12
Solubility Studies of Maropitant

| | | | Solubility | |
|---|---|---|---|---|
| | | Solvent | (mg/1 g of | |
| Test | Media | ratio | solvent) | Comments |
| 1 | Methanol | N/A | Up to 30 | Candidate to suspend |
| | | | | the API |
| 2 | Ethanol | N/A | Up to 15 | Candidate to suspend |
| | | | | the API |
| 3 | Isopropanol | N/A | insoluble | Candidate to suspend |
| | | | | the API |
| 4 | Acetone | N/A | Up to 120 | Candidate to |
| | | | | solubilize the API |
| 5 | MEK | N/A | Up to 150 | The best solvent to |
| | | | | solubilize the API |
| 6 | MEK:Maiscine | 40:2 | Up to 180 | |
| | CC | | | |
| 7 | MEK:Maiscine | 40:3 | Up to 220 | |
| | CC | | | |
| 8 | MEK:MCT | 40:2 | Up to 220 | MCT solubilize |
| 9 | MEK:MCT | 40:3 | Up to 240 | Maropitant better |
| | | | | than Maisine |
| 10 | MEK:PEG 300 | 40:2 | Up to 180 | As Maisine CC. PEG |
| | | | | 300 could be an option |
| | | | | as plasticizer and in |
| | | | | meantime a solubility |
| | | | | enhancer |
| 11 | MEK:MCT: | 40:3:2 | Up to 240 | No improvement |
| | PEG 300 | | | |
| 12 | MEK:Labrafil | 40:2 | Up to 220 | As MCT. Labrafil has |
| | 1944CS | | | an advantage to be |
| | | | | water dispersible |
| | | | | surfactant compare |
| | | | | to MCT (oily vehicle) |
| 13 | MEK:Labrafil | 40:3:2 | Up to 220 | No improvement |
| | 1944CS:MCT | | | |
| 14 | MEK:Labrafil | 40:3:2 | Up to 220 | No improvement |
| | 1944CS:PEG | | | |
| | 300 | | | |
| 15 | MEK:Glycerine | 40:2 | Up to 135 | Low solubility, gives |
| | | | | cloudy liquid |
| | | | | (compare to MEK |
| | | | | alone) |
| 16 | MEK:Tween 20 | 40:2 | Up to 180 | As Maisine CC & |
| | | | | PEG 300: advantage |
| | | | | to use tween 20 |
| | | | | instead of Maisine |
| | | | | since the former is |
| | | | | water soluble. |
| 17 | MEK:Methanol | 40:10 | Up to 155 | Methanol induce |
| | | | | reduction of solubility |
| | | | | but it is an option to |
| | | | | increase solvent |
| | | | | system polarity that |
| | | | | allow the introduction |
| | | | | of hydrophilic |
| | | | | ingredients as the film |
| | | | | former. |
| 18 | MEK:Methanol: | 40:10:3 | Up to 235 | No improvement |
| | MCT | | | compare to |
| | | | | MEK:MCT 40:3, but |
| | | | | MCT improve the |
| | | | | solubilisation in |
| | | | | MEK:Methanol 40:10. |
| 19 | MEK:Water | 40:4.5 | insoluble | Candidate to suspend |
| | | | | the API |
| 20 | MEK:Methanol: | 40:10:4:4 | Up to 317 | Best system to |
| | Labrafil | | | solubilize the API. |
| | 2125CCS: | | | Labrafil 2125CCS |
| | Tween 20 | | | Consists of mono-, di- |
| | | | | and triglycerides and |
| | | | | PEG-6 (MW 300) |
| | | | | mono- and diesters of |
| | | | | linoleic (C18:2) acid. |

Referring now to Table 12, it can be concluded that MCT and Labrafil 2125CS, Tween 20 solubilizing system are considered the optimal systems to formulate Maropitant film. Maropitant OF is developed following two approaches: as solubilized API in the film or suspended API within the oral film matrix.

According to some embodiments, the API is solubilized using MEK (or optimize the solubility in acetone or other combination solvent) and adding the solubilizing system (MCT and Labrafil 2125CS: Tween 20). The challenge is the limited number of film former that are soluble in MEK. To resolve that, we introduce ⅕ methanol in the solvent system to introduce some polarity to the solvent system. The solubility enhancers are introduced to promote the dissolution and to prevent the recrystallization of the API in vivo.

According to some embodiments, the API is suspended using one of the alcohols, water or a combination of those with MEK or acetone. The objective is to control the particle size of the API in the film blend. Homogenizer use is recommended to control the API PSD in the blend. Since the disclosed film formulation technology comprise a drying step to remove the solvents, this step introduce the risk of generating rapid crystallization when the API is solubilized in the blend. The use high TG film former polymers is preferred, though not required.

According to embodiments, the film forming polymers represent between 30% and 60%, preferably between 40% and 50% of the film dry weight, form. The desired ratio of API to solubilizers/surfactants is between 1:3 and 1:4.

In certain aspect of the present disclosure, the film matrix may disintegrate and/or dissolve in the physiological fluid. The modulation of the matrix breaking down involve pH control (Examples 143p80A 143P80B), surfactant HLB change (Examples 143P69A VS 143P80B), solubility of the active pharmaceutical ingredient or combination thereof.

TABLE 13
Formulation 11

| | | | | | | Weight |
|---|---|---|---|---|---|---|
| | Formulation | | Weight | % Wet | % Dry | (mg) |
| Order | 143P69A | Function | (g) | (wt/wt) | (wt/wt) | per Film |
| 4 | API | Responsible for | 6.00 | 8.43 | 31.28 | 60.00 |
| | | therapeutic effect | | | | |
| 3 | Hydroxylated | surfactant | 2.00 | 2.1 | 10.73 | 20.00 |
| | Lecithin | | | | | |
| 2 | Water | Solvent system | 52.00 | 73.05 | — | — |
| 1 | Glycerine | Co- | 0.94 | 1.32 | 4.90 | 9.40 |
| | | surfactant/plasticizer | | | | |
| 5 | HPC E4M | Suspending | 0.40 | 0.56 | 2.09 | 4.00 |
| | | agent/viscosity | | | | |
| | | increasing agent | | | | |
| 6 | HPC SSL | Amphiphilic solubility | 2.00 | 2.81 | 10.43 | 20.00 |
| | | enhancer | | | | |
| 7 | HPMC E50 | Agglomeration inhibitor | 0.64 | 0.90 | 3.34 | 6.40 |
| | | and mucoadhesive film | | | | |
| | | former | | | | |
| 8 | PEO 200K | Water soluble | 7.20 | 10.12 | 37.54 | 7200 |
| | | mucoadhesive film | | | | |
| | | former | | | | |

| | | | | |
|---|---|---|---|---|
| TOTAL | 71.18 | 100.0 | 100.0 | 191.80 |

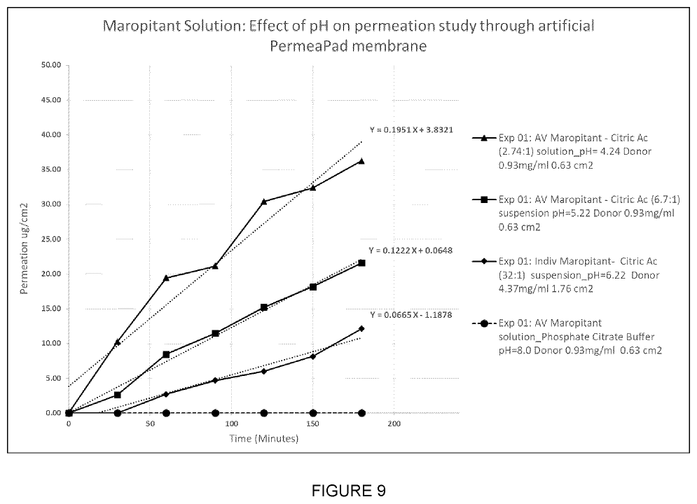

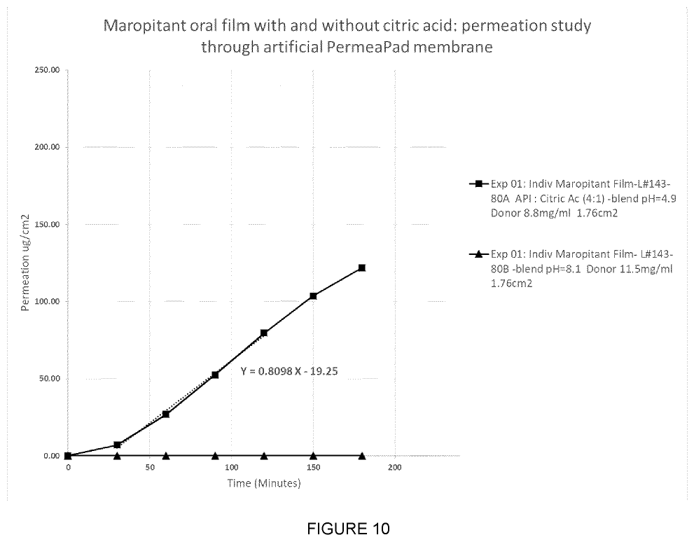

Numerous experiments were performed to improve the permeability of Maropitant through artificial membranes using the Franz cell model. Initially, solutions and suspensions of Maropitant and different acids at different ratios were prepared. FIG. 9 shows the permeability results of Maropitant/citric acid solution (ratio 2.74:1, pH: 4.24), Maropitant/citric acid suspension (ratio 6.7:1, pH: 5.22), Maropitant/citric acid suspension (ratio 32:1, pH: 6.22) and Maropitant/phosphate citrate buffer (pH: 8.0).

As shown in FIG. 9, results demonstrate that an acidic environment promotes Maropitant permeation.

Based on the results obtained with the solution/suspension of Maropitant at different pH, Maropitant oral films with and without citric acid were prepared.

TABLE 14
Formulation 12

| | Formulation | Weight | % Wet | % Dry | Weight (mg) |
|---|---|---|---|---|---|
| Order | 143P80A | (g) | (wt/wt) | (wt/wt) | per Film |
| 6 | API | 1.50 | 8.30 | 29.62 | 60.00 |
| 5 | Hydroxylated | 0.40 | 2.21 | 7.90 | 16.00 |
| | Lecithin | | | | |
| | Tween 20 | 0.10 | 0.55 | 1.97 | 4.00 |
| 4 | Citric acid | 0.37 | 2.05 | 7.31 | 14.80 |
| 3 | Water | 13.00 | 71.96 | — | — |
| 1 | Glycerine | 0.24 | 1.30 | 4.64 | 9.40 |
| 7 | HPMC 4M | 0.10 | 0.55 | 1.97 | 4.00 |
| 8 | HPC SSL | 0.50 | 2.77 | 9.87 | 20.00 |
| 9 | HPMC E50 | 0.16 | 0.89 | 3.16 | 6.40 |
| 10 | PEO 200K | 1.70 | 9.41 | 33.56 | 68.00 |

| | | | | |
|---|---|---|---|---|
| TOTAL | 18.07 | 100.0 | 100.0 | 202.60 |

TABLE 15
Formulation 13

| | Formulation | Weight | % Wet | % Dry | Weight (mg) |
|---|---|---|---|---|---|
| Order | 143P80B | (g) | (wt/wt) | (wt/wt) | per Film |
| 5 | API | 1.5 | 8.48 | 31.95 | 60.00 |
| 4 | Hydroxylated | 0.4 | 2.26 | 8.52 | 16.00 |
| | Lecithin | | | | |
| 2 | Tween 20 | 0.1 | 0.57 | 2.13 | 4.00 |
| 3 | Water | 13 | 73.47 | — | — |
| 1 | Glycerine | 0.235 | 1.33 | 5.01 | 9.40 |
| 7 | HPMC 4M | 0.1 | 0.57 | 2.13 | 4.00 |
| 8 | HPC SSL | 0.5 | 2.83 | 10.65 | 20.00 |
| 10 | HPMC E50 | 0.16 | 0.90 | 3.41 | 6.40 |
| 11 | PEO 200K | 1.7 | 9.61 | 36.21 | 68.00 |

| | | | | |
|---|---|---|---|---|
| TOTAL | 17.70 | 100.0 | 100.0 | 187.80 |

The preparation process of the film formulation with citric acid (Table 14) involves a water based system where the API is suspended in a surfactant, such as but not limited to Hydroxylated Lecithin, co-surfactants such as but not limited to Glycerine and Tween 20 and citric acid which are then homogenized. Once homogenization step is completed, the API suspension is stabilized with the addition of a high viscosity polymer, such as but not limited to HPMC (E4M). Once the polymer is added and homogenized, a low viscosity polymer such as but not limited to HPC (SSL) is added to the wet blend and mixed. The addition of the low viscosity polymer is followed by the addition of HPMC E50 to prevent API crystal growth in the final dry film. Subsequently, the main film former polymer such as but not limited to PEO is added and mixed in the blend. The final blend deaerated to remove the presence of air bubbles. Upon completion of the deaeration process, the blend is coated on a liner at specified gap, dried, cut to the required dimension and packaged in individual pouches.

The manufacture of the oral film without citric acid (Table 15) used the same manufacturing process as the film with citric acid.

A comparison of formulations 143P80A versus 143P80B reveals that the use of citric acid promotes erosion and the resulting OF erodes without disintegration.

A comparison of formulations 143P69A versus 143P80B (both do not include any citric acid) reveals that disintegration in the presence of tween 20 is slower.

The use of either citric acid or tween 20 appears to disrupt emulsion formation.

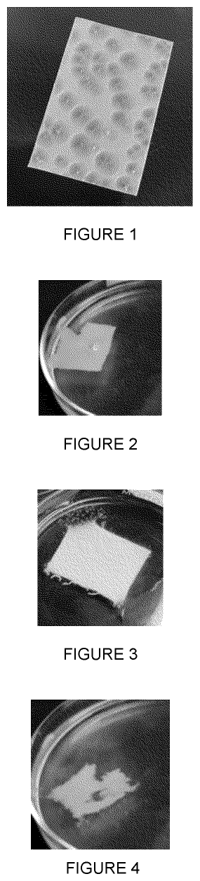

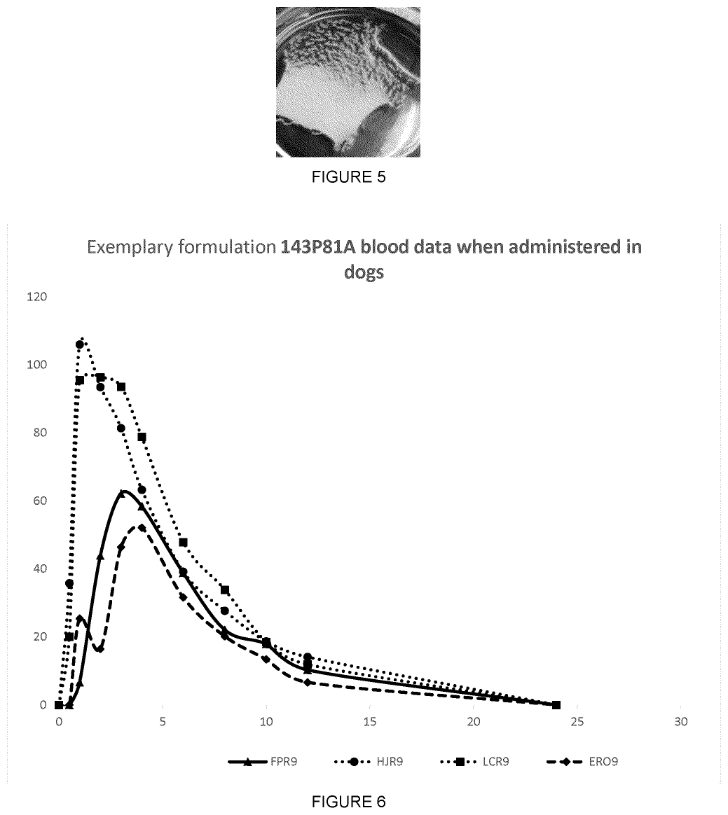

FIGS. 2 and 3 illustrate film 143P80A (FIG. 2) versus 143P80B (FIG. 3) in fake saliva pH 6,8 without shaking at 16 minutes. Similarly, FIGS. 4 and 5 illustrate film 143P80A (FIG. 4) versus 143P80B (FIG. 5) in fake saliva pH 6,87 without shaking at 21 minutes.

143P80B (FIG. 5) in fake saliva pH 6,8 without shaking at 21 minutes.

FIG. 9 illustrates improved Maropitant permeability when formulation includes citric acid versus without.

### Optimization of Mucoadhesion

OF must be designed to adhere to the surface of oral mucosa. The adhesion is assessed in comparison to a known mucoadhesive OF.

For the following examples (143P81A versus 143P81B), mucoadhesion was assessed by using swine skin gelatin dish plate to stimulate the oral mucosa, where 6.7% w/w gelatin in dissolved in deionized water, poured onto a Petri dish and allowed to cool at room temperature. A rectangular tested film is put in contact with the gelatin for 10 seconds and then detached. The tacking of the OF to the gelatin surface is assessed.

TABLE 16

| | | | | | | Weight |
|---|---|---|---|---|---|---|
| | Formulation | | Weight | % Wet | % Dry | (mg) |
| Order | 143P81A | | (g) | (wt/wt) | (wt/wt) | per Film |
| 4 | API | Responsible for | 1.50 | 8.11 | 27.30 | 60.00 |
| | | therapeutic effect | | | | |
| 3 | Hydorxylated | surfactant | 0.50 | 2.70 | 9.10 | 20.00 |
| | lecithin | | | | | |
| 2 | Water | Solvent system | 13.00 | 70.29 | — | — |
| 1 | Glycerine | Co- | 0.24 | 1.27 | 4.28 | 9.40 |
| | | surfactant/plasticizer | | | | |
| 5 | HPMC E4M | Suspending | 0.15 | 0.81 | 2.73 | 6.00 |
| | | agent/viscosity | | | | |
| | | increasing agent | | | | |
| 6 | HPC SSL | Amphiphilic solubility | 0.33 | 2.70 | 9.10 | 20.00 |
| | | enhancer | | | | |
| 7 | Sucralose | Sweetner | 0.20 | 1.08 | 3.64 | 8.00 |
| 9 | HPMC E50 | Agglomeration inhibitor | 0.33 | 0.87 | 2.91 | 6.40 |
| | | and mucoadhesive film | | | | |
| | | former | | | | |
| 10 | PEO 200K | Water soluble | 1.70 | 9.19 | 30.94 | 68.00 |
| | | mucoadhesive film | | | | |
| | | former | | | | |
| 8 | Savorboost | Flavor enhancer | 0.05 | 0.27 | 0.91 | 2.00 |
| | BK | | | | | |
| 8 | PC-0050 | Flavor | 0.30 | 1.62 | 5.46 | 12.00 |
| 8 | Magnasweet | Sweetner | 0.20 | 1.08 | 3.64 | 8.00 |

| | | | | |
|---|---|---|---|---|
| TOTAL | 18.50 | 100.0 | 100.0 | 219.80 |

TABLE 17

| | | | | | | Weight |
|---|---|---|---|---|---|---|
| | Formulation | | Weight | % Wet | % Dry | (mg) |
| Order | 143P81B | Function | (g) | (wt/wt) | (wt/wt) | per Film |
| 4 | API | Responsible for | 1.50 | 8.11 | 27.30 | 60.00 |
| | | therapeutic effect | | | | |
| 3 | Hydorxylated | surfactant | 0.50 | 2.70 | 9.10 | 20.00 |
| | lecithin | | | | | |
| 2 | Water | Solvent system | 13.00 | 70.29 | — | — |
| 1 | Glycerine | Co- | 0.24 | 1.27 | 4.28 | 9.40 |
| | | surfactant/plasticizer | | | | |
| 5 | HPMC E4M | Suspending | 0.15 | 0.81 | 2.73 | 6.00 |
| | | agent/viscosity | | | | |
| | | increasing agent | | | | |
| | HPC SSL | Amphiphilic solubility | 0.33 | 1.78 | 6.01 | 13.20 |
| | | enhancer | | | | |
| 6 | PEG 300 | Co- | 0.20 | 1.08 | 3.64 | 8.00 |
| | | surfactant/plasticizer | | | | |
| 9 | HPMC E50 | Agglomeration | 0.33 | 1.78 | 6.01 | 13.20 |
| | | inhibitor and | | | | |
| | | mucoadhesive film | | | | |
| | | former | | | | |
| 10 | PEO 200K | Water soluble | 1.70 | 9.19 | 30.94 | 68.00 |
| | | mucoadhesive film | | | | |
| | | former | | | | |
| 8 | Savorboost | Flavor enhancer | 0.05 | 0.27 | 0.91 | 2.00 |
| | BK | | | | | |
| 8 | PC-0050 | Flavor | 0.30 | 1.62 | 5.46 | 12.00 |
| 8 | Magnasweet | Sweetner | 0.20 | 1.08 | 3.64 | 8.00 |

| | | | | |
|---|---|---|---|---|
| TOTAL | 18.50 | 100.0 | 100.0 | 219.80 |

A comparison of formulations 143p81A (contains sugar: sucralose) versus 143P81B (contains plasticizer PEG300 and mucoadhesive polymer HPMC E50) reveals that 143p80A is more sticky.

TABLE 18
Exemplary formulation 143P81A blood data in dogs

| | Study | Exemplary formulation 143P81A (Strip C) | | | | | Strip |
|---|---|---|---|---|---|---|---|
| | Day | HR | FPR9 | HJR9 | LCR9 | ERO9 | C |
| ng/mL | −1 | 0 | BLOQ | BLOQ | BLOQ | BLOQ | 0 |
| | 0 | 0.5 | BLOQ | 35.8 | 20.1 | BLOQ | 27.95 |
| | 0 | 1 | 6.68 | 106 | 95.5 | 25.4 | 58.395 |
| | 0 | 2 | 43.9 | 93.5 | 96.3 | 16.5 | 62.55 |
| | 0 | 3 | 62.2 | 81.4 | 93.6 | 46.4 | 70.9 |
| | 0 | 4 | 58.4 | 63.3 | 78.8 | 52.1 | 63.15 |
| | 0 | 6 | 38.8 | 39.2 | 47.9 | 31.6 | 39.375 |
| | 0 | 8 | 22.1 | 27.7 | 33.9 | 20.2 | 25.975 |
| | 0 | 10 | 17.9 | 18.7 | 18.5 | 13.4 | 17.125 |
| | 0 | 12 | 10.3 | 14.1 | 11.9 | 6.66 | 10.74 |
| | 1 | 24 | BLOQ | BLOQ | BLOQ | BLOQ | 0 |

TABLE 19
CERENIA injectable blood data in dogs

| HR | KBR9 | JUR9 | ZAP9 | GBQ9 | Cerenia |
|---|---|---|---|---|---|
| 0 | BLOQ | BLOQ | BLOQ | BLOQ | 0.00 |
| 0.5 | 84.7 | 69.2 | 113 | 94.6 | 92.27 |
| 1 | 104 | 89.9 | 105 | 92.1 | 95.67 |
| 2 | 109 | 83.5 | 94.7 | 80.4 | 86.20 |
| 3 | 94.4 | 81.1 | 79.8 | 67.7 | 76.20 |
| 4 | 80.4 | 67.7 | 63.6 | 55.5 | 62.27 |
| 6 | 55.6 | 49.2 | 44.2 | 35.4 | 42.93 |
| 8 | 29.2 | 35.4 | 28.1 | 21.7 | 28.40 |
| 10 | 18.9 | 25.6 | 18.1 | 17.4 | 20.37 |
| 12 | 8.53 | 17 | 11.8 | 12.9 | 13.90 |
| 24 | BLOQ | BLOQ | BLOQ | BLOQ | 0.00 |

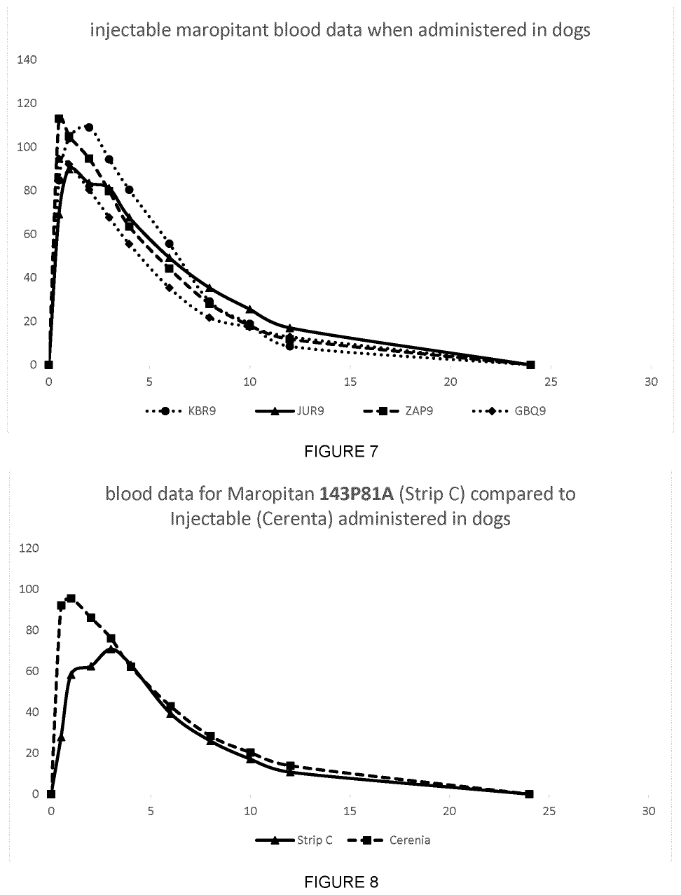

Referring now to Tables 18 and 19, it is disclosed blood data obtained by administering the exemplary formulation outlined in Table 16. The data of Tables 18 and 19 is shown in FIGS. 6, 7 and 8. Surprisingly, the disclosed high loading oral film dosage form exhibited a Cmax of up to more than 75% that of the injectable on average. The blood concentration an hour following administration was more than 50% that of the injectable, demonstrating the clear success of this animal administered high loading oral film formulation.

The formulation comprising tadalafil was compared to the formulation containing maropitant.

It is disclosed oral films with high API loading (60 mg) of one or more APIs of lipophilic, hydrophobic, hydrophilic, of one of the 4 classes of BCS classification

Tadalafil was also tested as a drug candidate. Tadalafil, taken by mouth, is used to treat male erectile dysfunction.

Tadalafil is practically insoluble in water. A formulation is developed in a mixture of organic solvents (methanol, isopropanol, and acetone).

TABLE 20

| | | Weight | % Wet | % Dry | Weight (mg) |
|---|---|---|---|---|---|
| Order | Excipient | (g) | (wt/wt) | (wt/wt) | per Film |
| 8 | API:Tadalafil | 9.30 | 3.10 | 11.10 | 59.97 |
| 5 | Sucralose | 0.53 | 0.18 | 0.63 | 3.42 |
| 5 | L-menthol | 0.96 | 0.32 | 1.15 | 6.19 |
| 5 | Ammonium | 0.48 | 0.16 | 0.57 | 3.10 |
| | glycyrrhizinate | | | | |
| 7 | Titanium dioxide | 2.09 | 0.70 | 2.49 | 13.48 |
| 9 | Plasdone K-90 | 36.76 | 12.26 | 43.86 | 237.06 |
| 7 | HPC Klucel GXF | 1.20 | 0.40 | 1.43 | 7.74 |
| 9 | HPC Klucel LF | 12.20 | 4.07 | 14.56 | 78.68 |
| 1 | PEG 600 | 3.30 | 1.10 | 3.94 | 21.28 |
| 2 | Labrafil M1944CS | 17.00 | 5.67 | 20.28 | 109.63 |
| 4 | Methanol | 29.20 | 9.74 | — | — |
| 4 | Isopropanol | 21.60 | 7.20 | — | — |
| 3 | Acetone | 165.3 | 55.11 | — | — |

| | | | | |
|---|---|---|---|---|
| TOTAL | 300 | 100.0 | 100.0 | 540.54 |

According to certain embodiments, the film matrix may also comprise pH modifiers, sweeteners, permeation enhancers, flavoring agents, plasticizers, film former polymers, opacifiers and solubility enhancers. The oral film may also comprise trace amounts of residual solvents. According to some embodiments, the flavoring agent may also act as a permeation enhancer.

According to certain embodiments, liquid film-forming formulations generally include at least one film-forming polymer and a solvent system. The solvent system can be comprised of a single solvent or a mixture of two or more solvents that are typically miscible. The liquid film-forming formulation may incorporate other ingredients that enhance or modify the functionality, processability, taste, stability, or aesthetics of the film. Such film-forming additives include colorants, opacifiers, flavorants, plasticizers, surfactants, stabilizer etc.

Water soluble polymers that can be employed in the disclosed films include water soluble cellulose derivatives, including hydroxypropylmethyl cellulose, hydroxyethyl cellulose, hydroxypropyl cellulose, polyvinyl pyrrolidone, copovidone (a copolymer of 1-vinyl-2-pyrrolidone and vinyl acetate), other copolymers of vinyl pyrrolidone, other polymers or copolymers of substituted vinyl pyrrolidone, derivatives of polyvinyl pyrrolidone, polyethylene oxide (PEO), carboxymethyl cellulose, polyvinyl alcohol, polysaccharides, natural gums, including xanthan, tragacanth, guar, acacia and arabic gums, and water soluble polyacrylates. As well, practically water insoluble polymers, such as microcrystalline cellulose, ethyl cellulose, and hypromellose phthalate can be used in the formulation. Combinations of these water soluble and non-water soluble polymers or other polymers can also be used.

According to certain embodiments, the film matrix may also comprise various weight ratios of API/polymer. The API/polymer weight ratios may vary depending on the active agent, however, should be close to 1. The polymer/API weight ratio of typical oral film is typically in the range of from about 2:1 to about 10:1 as opposed to what is disclosed herein where the ratio of API to polymer is close to 1:1 preferably in the range of about 0.8:1 to about 1:0.8 and more preferably in the range of about 0.9:1 to about 1:0.9. In other words, the disclosed oral film dosage forms have a polymer component that is close to identical from a weight standpoint.

The content of the above-mentioned poor water solubility API in an oral film dosage form preparation is generally 1 wt % to 40 wt %, preferably 2 wt % to 30 wt %, more preferably 5 wt % to 20 wt %.

According to certain embodiments, poorly water soluble APIs as outlined above are generally understood to be APIs categorized as a Class II or Class IV of the BCS. The poorly soluble compound or API may also be a salt of the API.

Examples of drugs that could be beneficially employed in the disclosed processes include aceclofenac, adenosine, adriamycin, alfacalcidol, alosetron, alprazolam, amoxacilline, amphetamine sulfate, aripiprazole, aspirin, atorvastatin calcium, atropine, bacitracin, bicalutamide, bosentan, budesonide, buspirone, carbamazepine, celecoxib, cilostazol, cisapride, citalopram, clofazimine, clopidogrel bisulfate, cyclosporin, cyproterone acetate, delta-9-tetrahydrocannabinol, danazol, delavirdine, desloratadine, dexamethasone, diazepam, diclofenac, dipyridamole, docetaxel, dolargin, domperidine, domperidone, donepezil, doxorubicin, efavirez, entacapone, estazolam, everolimus, ezetimibe, felodipine, flunitrazepam, flutamide, folic acid, fulvestran, furosemide gefitinib, gliperizide, griseofulvin, hydrocortisone, ibuprofen, indomethacin, itraconazone, ketoconazole, ketoprofen, landoprazole, lenalidomide, levonorgestrel, loperamide, loratadine, lovastatin, lysozyme, mecamylamine, metaphetamine, morphine, naproxen, naproxone, nifedipine, nitrazepam, norethindrone, norgestimate, norgestrel, ofloxacin, olanzepine, omeprazol, paclitaxel phytosterol, pimozide, piroxicam, prazepam, progesterone, raloxifene HCl, raloxifene, ridogrel, salicylic acid, simvastatin, stigmasterol, tadalafil, temsirolimus, terfenadine, tolvaptam, tracolimus, triclabendazole, trypsinsulin, tubocurarine, zidovudine ziprazidone, and ß-Estradiol.

According to certain embodiments, the oral film preparation having a high content of a poorly water-soluble API (e.g., oral film preparation wherein the content of a poorly water-soluble substance in the oral film preparation is not more than 20 wt %) is disclosed.

According to certain embodiments, the oral film dosage form comprises an API which is solubilized as opposed to being in particulate form within the film.

According to certain embodiments, the oral film dosage form comprises an API which is placed in suspension within the film matrix. In such embodiments, the API is a particulate API which preferably has particles with a particle size of D(90) between about 2-20 μm. Practically insoluble or poorly water soluble API may be incorporated with an oral film dosage form as a particulate active.

According to certain embodiments, the disclosed oral film formulation comprises a a suspending agent/viscosity increasing agent, a mucoadhesive film former, an amphiphilic solubility enhancer and a non-amphiphilic solubility enhancer, an agglomeration inhibitor, a surfactant, a co-surfactant where the suspending agent/viscosity increasing agent is present in a ratio to API content of between about 1:5 to about 1:15. The ratio of suspending agent/viscosity increasing agent to surfactant is generally between about 1:2.3 to about 1:6.5, preferably between about 1:3.5 to about 1:4.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The preferred agglomeration inhibitor in such a formulation is in a ratio to API content of between about 1:2.3 to about 1:16. The surfactant preferably present in a ratio to API content of between about 1:1 to about 1:4 and a co-surfactant and a surfactant in a ratio of between about 1:1 to about 1:3.25 and wherein the ratio of co-surfactant to API of between about 1:2.2 to about 1:7.

According to certain embodiments, the disclosed oral film formulation comprises a suspending agent/viscosity increasing agent, a mucoadhesive film former, an amphiphilic solubility enhancer and a non-amphiphilic solubility enhancer, an agglomeration inhibitor, a surfactant, a co-surfactant where the suspending agent/viscosity increasing agent is present in a ratio to API content of between about 1:5 to about 1:15, The ratio of suspending agent/viscosity increasing agent to surfactant is generally between about 1:2.3 to about 1:6.5, preferably between about 1:3.5 to about 1:4.5 and the ratio of viscosity increasing agent to surfactant is typically between about 1:2.3 to about 1:6.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The preferred agglomeration inhibitor in such a formulation is in a ratio to API content of between about 1:2.3 to about 1:16. The surfactant preferably present in a ratio to API content of between about 1:1 to about 1:4 and a co-surfactant and a surfactant in a ratio of between about 1:1 to about 1:3.25 and wherein the ratio of co-surfactant to API of between about 1:2.2 to about 1:7.

According to certain embodiments, the disclosed oral film formulation comprises a suspending agent/viscosity increasing agent, mucoadhesive film former, an amphiphilic solubility enhancer and a non-amphiphilic solubility enhancer, an agglomeration inhibitor, a surfactant, a co-surfactant where the suspending agent/viscosity increasing agent is present in a ratio to API content of between about 1:5 to about 1:15. The ratio of suspending agent/viscosity increasing agent to surfactant is generally between about 1:2.3 to about 1:6.5, preferably between about 1:3.5 to about 1:4.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The preferred agglomeration inhibitor in such a formulation is in a ratio to API content of between about 1:2.3 to about 1:16. The surfactant preferably present in a ratio to API content of between about 1:1 to about 1:4 and a co-surfactant and a surfactant in a ratio of between about 1:1 to about 1:3.25 and wherein the ratio of co-surfactant to API of between about 1:2.2 to about 1:7.

According to certain embodiments, the disclosed oral film formulation comprises a suspending agent/viscosity increasing agent, a mucoadhesive film former, an amphiphilic solubility enhancer and a non-amphiphilic solubility enhancer, an agglomeration inhibitor, a surfactant, a co-surfactant where the suspending agent/viscosity increasing agent is present in a ratio to the surfactant of between about 1:2.3 to about 1:6.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The preferred agglomeration inhibitor in such a formulation is in a ratio to API content of between about 1:2.3 to about 1:16. The surfactant preferably present in a ratio to API content of between about 1:1 to about 1:4 and a co-surfactant and a surfactant in a ratio of between about 1:1 to about 1:3.25 and wherein the ratio of co-surfactant to API of between about 1:2.2 to about 1:7.

According to certain embodiments, the disclosed oral film formulation comprises a suspending agent/viscosity increasing agent, a mucoadhesive film former, an amphiphilic solubility enhancer and a non-amphiphilic solubility enhancer, a surfactant, a co-surfactant where the suspending agent/viscosity increasing agent is present in a ratio to API content of between about 1:5 to about 1:15. The ratio of suspending agent/viscosity increasing agent to surfactant is generally between about 1:2.3 to about 1:6.5, preferably between about 1:3.5 to about 1:4.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The surfactant preferably present in a ratio to API content of between about 1:1 to about 1:4 and a co-surfactant and a surfactant in a ratio of between about 1:1 to about 1:3.25 and wherein the ratio of co-surfactant to API of between about 1:2.2 to about 1:7.

According to certain embodiments, the disclosed oral film formulation comprises a suspending agent/viscosity increasing agent, a mucoadhesive film former, an amphiphilic solubility enhancer and a non-amphiphilic solubility enhancer, an agglomeration inhibitor and a surfactant where the suspending agent/viscosity increasing agent is present in a ratio to the API of between about 1:5 to about 1:15. The ratio of suspending agent/viscosity increasing agent to surfactant is generally between about 1:2.3 to about 1:6.5, preferably between about 1:3.5 to about 1:4.5. The oral film formulation mucoadhesive film former to API ratio is between about 1:0.74 to about 1:1.5 while both amphiphilic solubility enhancer and non-amphiphilic solubility enhancer are present in a ratio of between about 9:1 to about 1:1 with a ratio of total solubility enhancer to API of between about 1:0.6 to 1:3, preferably between 1:1 to 1:2 with the non-amphiphilic solubility enhancer alone is in a ratio to API content of between about 0.03:1 to about 0.33:1. The preferred agglomeration inhibitor in such a formulation is in a ratio to API content of between about 1:2.3 to about 1:16. The surfactant preferably present in a ratio to API content of between about 1:1 to about 1:4.

In certain aspects of this disclosure, a process of making a product as disclosed includes a step of dispersing the active agent(s) in a dispersant/solubilizer to stabilize the amorphous form of the active agent(s) by inhibiting crystal nucleation and growth. A solid dispersion powder, comprising the active agent(s) in an amorphous form and a polymer dispersant, can be produced using known techniques. Examples of polymers that can be used as dispersants include cellulose acetate, cellulose acetate phthalate, copovidone, polyvinylpyrrolidone, ethylcellulose, eudragit E, eudragit NE, eudragit L & S, eudragit RL & RS, hydroxypropyl cellulose, hydroxyethylcellulose, hypromellose, hypromellose phthalate, hypromellose succinyl acetate, ethylene glycol, propylene glycol block copolymers (polaxamer), polyethylene glycol, polymethacrylates, polyvinyl acetate phthalate, polyvinyl caprolactam-polyvinyl acetate-polyethylene glycol graft copolymer and povidone.

The amorphous active agent(s) or solid dispersion containing the amorphous active agent(s) is mixed with the liquid film-forming formulation without dissolving the active agent(s). The suspension is mixed at a certain speed and for a limited time to produce a blend that has a uniformity of content of the suspended active agent(s), an acceptable low level of air bubbles, while maintaining the amorphous nature of the active agent(s). In addition, to increase the stability of the produced film, dispersants with known stability-enhancing effects by recrystallization prevention can be used. Stability of the oral film includes stability against the formation of degradation products over a defined period of time, as well as maintained amorphicity and resistance to heat. The stability of the oral film can be assessed by the dissolution profile of the active agent(s). Any decrease in the rate of dissolution is an indicator of amorphicity change of the active agent(s) within the film product.

The mixing speed in the wet blend is sufficient to introduce the amorphous or partially amorphous active agent(s) and create a wet blend suspension and final film product with uniformity of content as per definition in the United States Pharmacopeia (USP) test 905 and or ASTM E2810 or E2710. The mixing time is the time required in combination with the mixing speed to create a uniform suspended mixture that does not solubilize the active agent(s). If the amorphous active agent(s) dissolves in the liquid blend, the physical and chemical characteristics of the final product would change significantly, render the process unstable and unpredictable, and increase the chance of potential recrystallization. The wet blend mixing and its composition must be set in a way to prevent re-agglomeration leading to recrystallization of the amorphous active agent(s).

The method and process of making the oral film dosage form disclosed herein generally involves, preparing, obtaining, or otherwise providing an active agent(s) in solid amorphous form or partially amorphous form, optionally combining the active agent(s) with a polymer dispersant to obtain an active agent(s) polymer-based particle system referred as a solid dispersion, uniformly suspending the active agent(s) in a liquid film-forming formulation, and removing solvents (such as by evaporation in a drying oven) to obtain a solid oral film dosage form that contains the amorphous or partially amorphous active agent(s) uniformly and stably distributed in a film matrix.

### Manufacturing Procedure:

The oral film is produced by dissolving all the soluble ingredients and or dispersible ingredients in the media of choice and mixing until all excipients are solubilized and or dispersed yielding a uniform blend. The active ingredient is then added to the blend and solubilized or dispersed, depending on the formulation. The blend is mixed until get uniform and then polymers are added to the blend and mixed until completely dissolved. Once dissolved and homogenous, the mixing speed is lowered to allow degassing of the blend.

Preparation of a film product typically involves casting or otherwise thinly spreading the liquid film formulation on a substrate, drying (e.g., evaporating) all or most of the solvent(s) from the film to produce a thin, semi-solid/solid film sheet of material, and cutting the film sheet into individual unit dosage forms for packaging or processing.

The above description is considered that of the preferred embodiment(s) only. Modifications of these embodiments will occur by those skilled in the art and by those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film formulation comprising API, a suspending agent/viscosity increasing agent, a mucoadhesive film former, an amphiphilic solubility enhancer and a non-amphiphilic solubility enhancer, an agglomeration inhibitor and a surfactant, wherein the API content is at least 40 mg representing at least 22% of the total dry weight of the oral film.

2. The oral film formulation of claim 1, wherein the weight ratio of suspending agent/viscosity increasing agent to API is preferably between about 1:5 to about 1:15.

3. The oral film formulation of claim 1, wherein the weight ratio of mucoadhesive film former to API is between about 1:0.74 to about 1:1.5.

4. The oral film formulation of claim 1, wherein the weight ratio of amphiphilic solubility enhancer and non-amphiphilic solubility enhancer is between about 9:1 to about 1:1 with a weight ratio of total solubility enhancer to API being between about 1:0.6 to 1:3.

5. The oral film formulation of claim 1, wherein the weight ratio of agglomeration inhibitor to API content is between about 1:2.3 to about 1:16.

6. The oral film formulation of claim 1, wherein the weight ratio of surfactant to API is between about 1:1 to about 1:4.

7. The oral film formulation of claim 1, further comprising a co-surfactant.

8. The oral film formulation of claim 7, wherein the weight ratio of co-surfactant to surfactant is between about 1:1 to about 1:3.25 and wherein the weight ratio of co-surfactant to API is between about 1:2.2 to about 1:7.

9. The oral film formulation of claim 1, further comprising a preservative agent, wherein the weight ratio of preservative agent to API is preferably between about 1:200 to about 1:1333.

10. The oral film formulation of claim 1, further comprising a plasticizer.

11. The oral film formulation of claim 1, further comprising a flavor and a flavor enhancer.

12. The oral film formulation of claim 1, further comprising a sweetener.

13. The oral film formulation of claim 1, wherein the formulation is specifically adapted for use in animals.

14. An oral film formulation comprising a low solubility API, a surfactant, an acidic pH and at least two polymers, wherein the low solubility API is at least 22% by weight, preferably 30% by weight, most preferably 37% by weight of the oral film formulation.

15. The oral film formulation of claim 14, having a pH of less than 5, but preferably less than 4.

16. The oral film formulation of claim 14, wherein the low solubility API is Maropitant.

17. The oral film formulation of claim 14, further comprising a co-surfactant.

18. The oral film formulation of claim 14, further comprising a mucoadhesive film former.

19. The oral film formulation of claim 14, further comprising a flavor.

20. The oral formulation of claim 14, further comprising a flavor enhancer.

21. The oral film formulation of claim 14, further comprising a preservative agent.

22. The oral film formulation of claim 14, further comprising a sweetener.

23. The oral film formulation of claim 14, further comprising a plasticizer.

24. The oral film formulation of claim 14, wherein the formulation is specifically adapted for use in animals.
